(19) United States
(12) Patent Application Publication
(10) Pub. No.: US 2014/0276107 A1
(43) Pub. Date: Sep. 18, 2014

(54) PHOTODYNAMIC DIAGNOSIS

(71) Applicant: Photocure ASA, Oslo (NO)

(72) Inventor: Ketil Widerberg, Oslo (NO)

(21) Appl. No.: 14/351,770

(22) PCT Filed: Oct. 12, 2012
(86) PCT No.: PCT/EP2012/070346

§ 371 Date: Apr. 14, 2014

(30) Foreign Application Priority Data
Oct. 14, 2011 (EP) 11185253.9
Oct. 14, 2011 (EP) 11185256.2

(51) Int. Cl. A61K 49/00; A61B 5/00

(52) U.S. Cl. CPC A61K 49/0052; A61B 5/0071
USPC 600/476

(57) ABSTRACT

This invention relates to the diagnosis of abnormalities of the epithelial-lined surface of the esophagus and, in particular, to the use of esters of 5-aminolevulinic acid (5-ALA) in methods of photodynamic diagnosis (PDD) of Barrett's esophagus and in diagnostic methods which monitor the progression of the disease with high specificity.

This invention relates generally to the diagnosis of abnormalities of the epithelial-lined surface of the esophagus. More particularly, it relates to the use of esters of 5-aminolevulinic acid (5-ALA) in methods of photodynamic diagnosis of Barrett's esophagus and in diagnostic methods for monitoring the progression of the disease.

Esophageal cancer includes both squamous cell carcinoma and adenocarcinoma. The incidence of esophageal adenocarcinoma has increased by almost 400% during recent decades and it is now the cancer with the fastest rising incidence in the Western world. Although it can be treated, for example by surgery or chemotherapy, it is rare that the disease can be cured; the mortality rate for those diagnosed with the disease is over 85%. Early diagnosis is therefore critical in order to improve survival rates.

Most esophageal adenocarcinomas are considered to arise from the condition known as ‘Barrett's esophagus’. Barrett's esophagus is a condition in which the normal squamous lining of the distal esophagus is replaced by columnar epithelium with intestinal metaplasia. It may be recognised endoscopically by a salmon-coloured mucosa above the lower esophageal sphincter that separates the stomach from the esophagus (a normal esophagus is usually light pink in colour). The condition typically arises as a complication of gastro-esophageal reflux disorder (GERD) which causes the normal cells that line the esophagus (squamous cells) to turn into specialised columnar cells that resemble those found in the intestinal epithelium (‘intestinal metaplasia’).

Intestinal metaplasia is a pre-malignant condition that, through a series of cellular changes, may develop further into low-grade dysplasia, high-grade dysplasia and, ultimately, adenocarcinoma of the esophagus. Dysplasia is not usually visible when using standard endoscopic surveillance methods and can remain undetected until the invasive adenocarcinoma stage. The ability to monitor the various stages of the disease is further complicated by the simultaneous presence of metaplasia as well as dysplasia and early cancer which are difficult to differentiate with standard endoscopy techniques. The ability not only to diagnose Barrett's esophagus at an early stage, but also to monitor its progression is key to preventing esophageal cancer and to improving survival rates.

Currently, Barrett's esophagus is diagnosed and its progression monitored by taking random four-quadrant biopsies (RFQB) for every 1-2 cm length of the esophagus followed by histopathologic assessment of the biopsy specimens (Bartelsman et al. Eur. J. Cancer Prevention, 1: 323-325, 1992). Patients with known intestinal metaplasia undergo regular follow-up endoscopy with RFQB. This procedure increases the rate of early diagnosis of adenocarcinomas, but investigates only a small proportion of the epithelial surface which may be at risk; it is therefore associated with sampling errors (note that dysplastic changes often occur in a spatially heterogeneous fashion). Other drawbacks are that the method is costly, time-consuming to perform, and generally uncomfortable for the patient.

Alternative techniques for improved detection of intraepithelial dysplasia in Barrett's esophagus have been proposed. Endoscopic fluorescence detection following administration and photoactivation (i.e. activation with light) of 5-aminolevulinic acid (5-ALA) has been investigated. After systemic administration of 5-ALA and uptake into cells, it is converted intracellularly to the photosensitizer protoporphyrin IX (PpIX). Upon photoactivation, i.e. exposure of the area of interest to light, PpIX is excited and displays in response a fluorescence which is detected. This technique is known as photodynamic diagnosis (also herein referred to as ‘PDD’). The technique is based on the principle that the photosensitizer accumulates preferentially in metabolically active tissue, such as dysplastic or cancerous tissue; hence such tissue can be distinguished from healthy tissue. The mechanisms are still not fully understood, but studies suggest that accumulation is not due to selective uptake of 5-ALA by pre-cancerous or cancerous cells. Rather, there are similar levels of uptake in all cell types, but the processes of conversion and elimination are different in metabolically active cells, leading to a concentration gradient between abnormally proliferating cells and normal tissue.

Previous studies have shown that 5-ALA can distinguish high-grade dysplastic from non-dysplastic epithelial tissue in Barrett's esophagus (Brand et al., Gastrointestinal Endoscopy 56(4): 479-487, 2002). However high levels of background autofluorescence can diminish the contrast between dysplastic and non-dysplastic tissue.

Further studies have been carried out to assess the performance of 5-ALA in endoscopic fluorescence detection of intraepithelial dysplasia, and early stage cancers in Barrett's esophagus (Stepinac et al., Endoscopy 35(8): 663-668, 2003; Endlicher et al., Gut 48: 314-319, 2001). These have shown that sensitivity for detection of dysplastic tissue increases when using higher concentrations of 5-ALA, especially after systemic application of 5-ALA at 20 mg/kg and 30 mg/kg. However, specificity was found to be poor under these conditions (i.e. there was a high rate of false positive fluorescence). Systemic application of 20 mg/kg or more of 5-ALA is thus not recommended due to low specificity and increasing side effects at high dosages. It has been suggested that the high rate of false positives found in these earlier studies mainly arises due to the higher drug dose which induces a disturbing background fluorescence (i.e. noise) from inflamed, albeit otherwise healthy, mucosal tissues. Other limiting factors responsible for false positive fluorescence when using 5-ALA include the presence of metaplastic tissue which also induces strong fluorescence. Stepinac et al. (2003) also found that although endoscopic fluorescence detection using 5-ALA is effective in detecting high-grade intraepithelial neoplasia and adenocarcinoma, it is not accurate in detecting low-grade neoplastic tissue, which is likely to develop into cancerous tissue.

WO 2009/109569 suggests the use of esters of 5-ALA, specifically 5-ALA hexyl ester, for detecting early stage dysplasia in the esophagus and esophageal cancer.

There thus remains a need for alternative methods for detecting and monitoring abnormal changes of the epithelial cells in Barrett's esophagus which are reliable, cost-effective and simple to carry out. In particular, a need exists for such methods with improved sensitivity and which enable a clearer demarcation to be made between the various stages of the disease from early stage metaplasia through to later dysplastic and neoplastic states. This will enable earlier detection of any changes in the epithelial cells of the esophagus which may lead to pre-cancerous conditions or to esophageal cancer, e.g. esophageal adenocarcinomas.

We have now found that 5-ALA esters are particularly suitable for use in photodynamic methods for diagnosing the early stages of Barrett's esophagus (metaplasia) and, in particular, in methods of differentiating later-stage (e.g. pre-cancerous) abnormalities in the esophageal surface, i.e. in enhancing the contrast between normal/Barrett's tissue on the one hand and pre-cancerous and cancerous tissue on the other.

5-ALA esters are already known to have a number of advantages over 5-ALA itself, such as faster penetration into tissues; better enhancement of PpIX production; improved selectivity for the target tissue, etc. However, what we have now surprisingly found in the context of PDD in the esophagus is that, compared to 5-ALA, the use of 5-ALA esters provides an enhanced fluorescence contrast between dysplastic, neoplastic and cancer tissue and metaplastic/normal tissue. The reduction in background noise from both normal and metaplastic tissues when using the 5-ALA esters provides for superior demarcation of dysplastic, neoplastic and cancer tissues. By enhancing the detection of later stage abnormalities in the esophagus using 5-ALA esters, high-risk areas can more readily be identified and at a much earlier stage, leading to the potential for less invasive and safer treatment, e.g. by operative intervention or chemotherapy. This represents a significant advance in the ability to diagnose and treat later stage malignancies before the onset of cancer.

These findings are not foreshadowed in any of the current literature. Although bioadhesive hydrogels have been investigated for topical delivery of 5-ALA hexyl ester to Barrett's esophagus (Collaud et al., Journal of Controlled Release 23: 203-210, 2007), this earlier study was carried out on healthy volunteers and with the sole aim of determining the best esophageal adhesion and release profiles of the active. Visualization of the mucoadhesive properties of the formulations was carried out using a blue dye incorporated into the formulations as a contrast agent. No fluorescence diagnosis was performed.

Viewed from one aspect, the invention provides a method of photodynamic diagnosis of an abnormality of the epithelial lining of the esophagus, said method comprising the following steps:
(a) administering to a subject, e.g. a human or non-human animal, a 5-ALA ester, or a pharmaceutically acceptable salt thereof;
(b) if necessary, waiting for a time period necessary for the 5-ALA ester or pharmaceutically acceptable salt thereof to be converted into a photosensitizer and achieve an effective tissue concentration at the desired target site in the esophagus;
(c) exposing the epithelial lining of the esophagus to light whereby to photoactivate the photosensitizer; and
(d) detecting a fluorescence intensity indicative of said abnormality.

In carrying out the diagnostic method of the invention, differences in fluorescence intensity from the different tissue types (i.e. both normal and abnormal, e.g. normal, metaplastic, dysplastic, neoplastic and cancerous) present at the target site may be detected. It is these differences in fluorescence intensity which may be used to indicate the presence or absence of any abnormality within the target area.

In a further aspect the invention provides a 5-ALA ester, or a pharmaceutically acceptable salt thereof, for use in a method of photodynamic diagnosis as herein described.

The use of a 5-ALA ester, or a pharmaceutically acceptable salt thereof, in the manufacture of a medicament for use in a method of photodynamic diagnosis as herein described forms a further aspect of the invention.

As used herein, the term “diagnosis” encompasses not only the detection (i.e. identification) of an abnormality, but also monitoring or surveillance of its progression and development. Abnormalities of the epithelial lining of the esophagus encompass not only cancerous conditions but also conditions which may be considered pre-cancerous or non-cancerous.

The term “pre-cancerous condition” denotes a tissue abnormality which, if left untreated, may ultimately lead to cancer. It is a generalized state associated with a significantly increased risk of cancer. A pre-cancerous condition may manifest itself by extensive and/or abnormal proliferation of cells, e.g. dysplasia and/or neoplasia.

Cancerous conditions in the esophagus include basal cell carcinomas and adenocarcinomas.

The term “non-cancerous conditions” includes tissue abnormalities with no or low malignant potential such as metaplasia.

In a preferred embodiment the invention relates to the diagnosis of Barrett's esophagus. As used herein, the term “Barrett's esophagus” is intended to encompass the presence of metaplastic epithelium. In later stage Barrett's esophagus, the presence of dysplastic tissue may be observed.

The term “metaplastic” denotes cells or tissues which have been reversibly replaced by another differentiated cell type. Metaplastic tissue is considered non-carcinogenic. The term “dysplastic” denotes the abnormal development or growth of cells or tissues. The term “neoplastic” denotes the abnormal proliferation of cells or tissues. Both the presence of dysplastic and neoplastic tissues is considered a pre-cancerous condition.

The term “5-ALA ester” denotes esters of 5-aminolevulinic acid (i.e. 5-amino-4-oxo-pentanoic acid).

The term “pharmaceutically acceptable salt” denotes a salt that is suitable for use in a pharmaceutical product and which fulfills the requirements related to for instance safety, bioavailability and tolerability (see for instance P. H. Stahl et al. (eds.) Handbook of Pharmaceutical Salts, Publisher Helvetica Chimica Acta, Zurich, 2002).

The invention encompasses the photodynamic diagnosis of early stage Barrett's esophagus, i.e. detection of the presence of metaplastic cells, using the 5-ALA esters herein described. Once metaplasia has been detected, these 5-ALA esters may also be used in photodynamic methods for monitoring the progression of the disease, for example in detecting early and/or late stage dysplasia. Monitoring of patients with Barrett's esophagus at regular intervals, e.g. annually or bi-annually, enables early detection of any progression of the disease to pre-cancerous (e.g. dysplastic and/or neoplastic) and/or cancerous (e.g. adenocarcinoma) stages. As will be appreciated, early identification of areas of the esophagus which are at high-risk of developing into cancer improves the chances of successful treatment, e.g. by operative intervention.

Detection of fluorescence may be used to identify the presence of certain abnormal cell or tissue types, for example, any of metaplastic, dysplastic or neoplastic cells or tissues. Alternatively, measurement of fluorescence intensity from the esophagus may be used to distinguish between the different types of cells or tissues which may be present. Identification of any high-risk areas (e.g. those comprising dysplastic or neoplastic cells) for operative intervention forms a preferred aspect of the invention.

The results from any of the diagnostic methods which are described herein may also provide a useful guide for any subsequent biopsy and/or as a guide to the areas for subsequent treatment. In one embodiment, the methods of diagnosis may therefore be performed prior to carrying out a biopsy and/or therapeutic treatment.

The use of 5-ALA esters both in methods of photodynamic therapy (PDT) and photodynamic diagnosis (PDD) is well known in the scientific and patent literature (see, for example, WO 2006/051269, WO 2005/092838, WO 03/011265, WO 02/09690, WO 02/10120, WO 2003/041673 and U.S. Pat. No. 6,034,267, the contents of which are incorporated herein by reference). All such 5-ALA esters and their pharmaceutically acceptable salts are suitable for use in the diagnostic methods herein described.

The 5-ALA esters useful in accordance with the invention may be any ester of 5-ALA capable of forming protoporphyrins, e.g. PpIX or a PpIX derivative (e.g. a PpIX ester) in vivo. Typically, such esters will be a precursor of PpIX or of a PpIX derivative in the biosynthetic pathway for haem and which are therefore capable of inducing an accumulation of PpIX following administration in vivo.

Esters of 5-ALA which are N-substituted may be used in the invention. However, those compounds in which the 5-amino group is unsubstituted are particularly preferred. Such compounds are generally known and described in the literature; see, for example, WO 96/28412 and WO 02/10120 to Photocure ASA, the contents of which are incorporated herein by reference.

Esters resulting from a reaction of 5-ALA with substituted or unsubstituted alkanols, i.e. alkyl esters and substituted alkyl esters, and pharmaceutically acceptable salts thereof, are especially preferred for use in the invention. Examples of such compounds include those of general formula I and pharmaceutically acceptable salts thereof:

R²₂N—CH₂COCH₂—CH₂CO—OR¹ (I)

wherein

R¹ represents an optionally substituted alkyl group; and

R² each independently represents a hydrogen atom or a group R¹.

As used herein, the term “alkyl”, unless stated otherwise, includes any long or short chain, cyclic, straight-chained or branched, saturated or unsaturated aliphatic hydrocarbon group. Unsaturated alkyl groups may be mono- or polyunsaturated and include both alkenyl and alkynyl groups. Unless stated otherwise, such alkyl groups may contain up to 40 carbon atoms. However, alkyl groups containing up to 30 carbon atoms, preferably up to 10, particularly preferably up to 8, especially preferably up to 6 carbon atoms, are preferred.

In the compounds of formula I, the R¹ groups are substituted or unsubstituted alkyl groups. If R¹ is a substituted alkyl group, one or more substituents are either attached to the alkyl group and/or interrupt the alkyl group. Suitable substituents that are attached to the alkyl group are those selected from hydroxy, alkoxy, acyloxy, alkoxycarbonyloxy, amino, aryl, nitro, oxo, fluoro, —SR³, —NR³₂ and —PR³₂, wherein R³ is a hydrogen atom or a C₁₋₆ alkyl group. Suitable substituents that interrupt the alkyl group are those selected from —O—, —S— or —PR³ (where R³ is as hereinbefore defined).

In one embodiment, R¹ may be an alkyl group substituted with one or more aryl substituents, i.e. aryl groups, preferably an alkyl group substituted with one aryl group.

As used herein, the term “aryl group” denotes an aromatic group which may or may not contain heteroatoms like nitrogen, oxygen or sulfur. Aryl groups which do not contain heteroatoms are preferred. Preferred aryl groups comprise up to 20 carbon atoms, more preferably up to 12 carbon atoms, for example, 10 or 6 carbon atoms. Preferred embodiments of aryl groups are phenyl and naphthyl, especially phenyl. Further, the aryl group may optionally be substituted by one or more, more preferably one or two, substituents. Preferably, the aryl group is substituted at the meta- or para-position, most preferably the para-position. Suitable substituents include haloalkyl (e.g. trifluoromethyl), alkoxy (preferably alkoxy groups containing 1 to 6 carbon atoms), halo (e.g. iodo, bromo, chloro or fluoro, preferably chloro or fluoro), nitro and C₁₋₆ alkyl (preferably C₁₋₄ alkyl). Preferred C₁₋₆ alkyl groups include methyl, isopropyl and t-butyl, particularly methyl. Particularly preferred aryl substituents are chloro and nitro. However, still more preferably the aryl group is unsubstituted.

Preferred such aryl-substituted R¹ groups include benzyl, 4-isopropylbenzyl, 4-methylbenzyl, 2-methylbenzyl, 3-methylbenzyl, 4-[t-butyl]benzyl, 4-[trifluoromethyl]benzyl, 4-methoxybenzyl, 3,4-[di-chloro]benzyl, 4-chlorobenzyl, 4-fluorobenzyl, 2-fluorobenzyl, 3-fluorobenzyl, 2,3,4,5,6-pentafluorobenzyl, 3-nitrobenzyl, 4-nitrobenzyl, 2-phenylethyl, 4-phenylbutyl, 3-pyridinyl-methyl, 4-diphenyl-methyl and benzyl-5-[(1-acetyloxyethoxy)-carbonyl]. More preferred such R¹ groups are benzyl, 4-isopropylbenzyl, 4-methylbenzyl, 4-nitrobenzyl and 4-chlorobenzyl. Most preferred is benzyl.

If R¹ is a substituted alkyl group, one or more oxo substituents are preferred. Preferably, such groups are straight-chained C₄₋₁₂ alkyl groups which are substituted by one or more oxo groups, preferably by one to five oxo groups. The oxo groups are preferably present in the substituted alkyl group in an alternating order, i.e. resulting in short chain polyethylene glycol substituents. Preferred examples of such groups include 3,6-dioxa-1-octyl and 3,6,9-trioxa-1-decyl. In another preferred embodiment, R¹ is an alkyl group interrupted by one or more oxygen atoms (ether or polyether group), preferably a straight-chained C₄₋₁₂ alkyl and more preferably a straight-chained C₆₋₁₀ alkyl group being interrupted by 1 to 4 oxygen atoms, more preferably a straight-chained polyethylene glycol group (—(CH₂)₂—O—)ₙ with n being an integer of from 1 to 5.

If R¹ is an unsubstituted alkyl group, R¹ groups that are saturated straight-chained or branched alkyl groups are preferred. If R¹ is a saturated straight-chained alkyl group, C₁₋₁₀ straight-chained alkyl groups are preferred. Representative examples of suitable straight-chained alkyl groups include methyl, ethyl, n-propyl, n-butyl, n-pentyl, n-hexyl and n-octyl. Particularly preferred are C₁₋₆ straight-chained alkyl groups. Most particularly preferred is n-hexyl. If R¹ is a saturated branched alkyl group, such branched alkyl groups preferably consist of a stem of 4 to 8, preferably 5 to 8 straight-chained carbon atoms and said stem is branched by one or more C₁₋₆ alkyl groups, preferably C₁₋₂ alkyl groups. Examples of such saturated branched alkyl groups include 2-methylpentyl, 4-methylpentyl, 1-ethylbutyl and 3,3-dimethyl-1-butyl.

In the compounds of formula I, each R² independently represents a hydrogen atom or a group R¹. Particularly preferred for use in the invention are those compounds of formula I in which at least one R² represents a hydrogen atom. In especially preferred compounds each R² represents a hydrogen atom.

Preferred 5-ALA esters for use in the invention include methyl ALA ester, ethyl ALA ester, propyl ALA ester, butyl ALA ester, pentyl ALA ester, hexyl ALA ester, octyl ALA ester, 2-methoxyethyl ALA ester, 2-methylpentyl ALA ester, 4-methylpentyl ALA ester, 1-ethylbutyl ALA ester, 3,3-dimethyl-1-butyl ALA ester, benzyl ALA ester, 4-isopropylbenzyl ALA ester, 4-methylbenzyl ALA ester, 2-methylbenzyl ALA ester, 3-methylbenzyl ALA ester, 4-[t-butyl]benzyl ALA ester, 4-[trifluoromethyl]benzyl ALA ester, 4-methoxybenzyl ALA ester, 3,4-[di-chlorobenzyl ALA ester, 4-chlorobenzyl ALA ester, 4-fluorobenzyl ALA ester, 2-fluorobenzyl ALA ester, 3-fluorobenzyl ALA ester, 2,3,4,5,6-pentafluorobenzyl ALA ester, 3-nitrobenzyl ALA ester, 4-nitrobenzyl ALA ester, 2-phenylethyl ALA ester, 4-phenylbutyl ALA ester, 3-pyridinyl-methyl ALA ester, 4-diphenyl-methyl ALA ester and benzyl-5-[(1-acetyloxyethoxy)-carbonyl]amino levulinate, and 3-methylbenzyl ALA ester.

The 5-ALA esters for use in the invention may be in the form of a free amine, e.g. —NH₂, —NHR² or —NR²R² or preferably in the form of a pharmaceutically acceptable salt. Such salts preferably are acid addition salts with pharmaceutically acceptable organic or inorganic acids. Suitable acids include, for example, hydrochloric, nitric, hydrobromic, phosphoric, sulfuric, sulfonic and sulfonic acid derivatives (the salts of ALA-esters and the latter acids are described in WO 2005/092838 to Photocure ASA, the entire contents of which are incorporated herein by reference). A preferred acid is hydrochloride acid, HCl. Further preferred acids are nitric acid, sulfonic acid and sulfonic acid derivatives (e.g. mesylate or tosylate). Procedures for salt formation are conventional in the art and are for instance described in WO 2005/092838.

Preferred compounds of formula I and pharmaceutically acceptable salts thereof for use in the invention are those wherein R¹ represents an unsubstituted alkyl group, preferably an unsubstituted , saturated, straight-chained or branched, alkyl group (e.g. a C₁₋₁₀ alkyl group). Particularly preferred compounds are those wherein R¹ is hexyl, more preferably n-hexyl, and both R² represent hydrogen, i.e. 5-ALA hexyl ester and pharmaceutically acceptable salts thereof. The preferred compound for use in the invention is 5-ALA hexyl ester and its pharmaceutically acceptable salts, preferably the hydrochloride salt, nitrate salt or sulfonic acid salts or sulfonic acid derivative salts, such as mesylate, tosylate or napsylate.

5-ALA esters and pharmaceutically acceptable salts thereof for use in the invention may be prepared by any conventional procedure available in the art, e.g. as described in WO 96/28412, WO 02/10120, WO 03/041673 and in N. Fotinos et al., Photochemistry and Photobiology 2006: 82: 994-1015 and the cited literature references therein. Briefly, 5-ALA esters may be prepared by reaction of 5-ALA with the appropriate alcohol in the presence of a catalyst, e.g. an acid. Pharmaceutically acceptable salts of 5-ALA esters may be prepared as described hereinbefore by reaction of a pharmaceutically acceptable 5-ALA salt, e.g. 5-ALA hydrochloride with the appropriate alcohol. Alternatively compounds for use in the invention like 5-ALA hexyl ester may be available commercially, e.g. from Photocure ASA, Norway.

The compounds for use according to the invention may be formulated in any conventional manner with one or more physiologically acceptable carriers or excipients, according to techniques well known in the art. Where appropriate, the compounds or compositions according to the invention are sterilized, e.g. by y-irradiation, autoclaving or heat sterilization, before or after the addition of a carrier or excipient where that is present, to provide sterile formulations.

Compositions for use in the invention may be administered systemically (e.g. orally or parenterally), but preferably these will be administered locally. Local administration may be achieved by injection or, more preferably, by topical application (e.g. by local instillation) at or near the desired target site. Administration may also be carried out distally at the upper end of the esophagus, e.g. by oral administration. As will be described herein, oral administration of certain formulations (e.g. those which are sufficiently viscous or which become so on contact with the mucosal surfaces of the esophagus) may be employed to achieve direct, local application of the photosensitizing agent to the intended site or sites of the esophageal surface. Suitable formulations include those described in WO 2009/109569, the contents of which are herein incorporated by reference.

Topical administration involving direct contact of the composition with the intended site or sites in the epithelial surface of the esophagus may be achieved by techniques known in the art, e.g. by the use of catheters (e.g. balloon catheters), stents or other appropriate drug delivery systems. A stent which can be used to topically administer the composition is described later in this application. Endoscopic methods may, for example, be employed. For direct topical application using such methods, particularly when using a catheter, it is preferred that the viscosity of the composition will be sufficiently low to permit its application. For example, when using a catheter, the composition will usually be in the form of a solution.

Suitable formulations for topical application include gels, sprays, powders, films, aerosols, solutions and any other conventional pharmaceutical forms in the art. One example of a product which is in the form of a solution and which may be used in the invention is Hexvix®. Developed by Photocure ASA (Oslo, Norway), this product comprises 5-ALA hexyl ester. Hexvix® is an aqueous solution of 5-ALA hexyl ester which is freshly prepared on site from a lyophilized powder of the 5-ALA hexyl ester and a dissolution medium. Hexvix® may be instilled or, more preferably, sprayed onto the esophagus.

Gel formulations may be used in the invention. These may contain the 5-ALA ester formulated with, for example, an aqueous or oily base with the addition of suitable thickening and/or gelling agents. Any thickening or gelling agents used should be non-toxic, non-irritant and devoid of leachable impurities. They should be inert towards the active ingredients, i.e. should not promote its degradation.

Powders for use in the invention may be formed with the aid of any suitable powder base. Sprays and solutions may be formulated with an aqueous or non-aqueous base also comprising one or more dispersing, solubilising or suspending agents. Aerosol sprays are conveniently delivered from pressurised packs with the use of a suitable propellant.

The compositions may additionally include lubricating agents, wetting agents, emulsifying agents, suspending agents, preserving agents, flavouring agents, odour enhancers and/or adsorption enhancers, e.g. surface penetrating agents as mentioned below, and the like. Solubilizing and/or stabilizing agents may also be used, e.g. cyclodextrins (CD) α, β, γ and HP-cyclodextrin.

Preferably, the 5-ALA ester is released from the composition on contact with the mucosa-lined surface of the esophagus. The compositions may be formulated so as to provide rapid release of the 5-ALA ester after administration to the subject by employing procedures well known in the art. Preferred are compositions formulated for release of the 5-ALA ester within a period of from 15 minutes to 1 hour, preferably 20 to 45 minutes, following administration.

It is desirable that the compositions should maintain contact with the esophageal surface for a sufficient period to permit the uptake of the 5-ALA esters. One way in which this may be achieved is through the use of a gelling agent. Examples of suitable gelling agents include hydrogels and in-situ gelling agents such as thermosetting gels (or thermoreversible gels). One embodiment of a thermosetting gel is one which is liquid at ambient temperature (e.g. 25° C.) and more viscous at body temperature (e.g. 37° C.).

Examples of suitable hydrogels include polyacrylic hydrogels, cellulose polymers (e.g. carboxymethylcellulose (CMC) polymer), and the like.

The ability to control the rate of release of the 5-ALA ester from the formulations herein described is particularly desirable. Controlled release includes rapid release as well as prolonged or sustained release, although generally it will be desirable that this should be delivered rapidly on contact with the mucosal surface. One way in which controlled release may be achieved is by appropriate selection of a bioadhesive, for example by selecting a bioadhesive which is capable of providing delayed (sustained) release of the active component of the formulation. Bioadhesive systems suitable for use in this regard are generally known and described in the art and include, in particular, the polymeric bioadhesives herein described. Such systems can be adjusted to control the release rate of active by varying the amount of cross-linking agent in the polymer. A suitable embodiment of a polymer for use in this regard is Polycarbophil which is commercially available from B. F. Goodrich under the tradename Noveon®-AA1. Polycarbophil is a polyacrylic acid cross-linked with divinyl glycol.

The compositions for use in the invention may also be provided in a form adapted for oral or parenteral administration, for example by intravenous injection. Alternative pharmaceutical forms thus include plain or coated tablets, capsules, suspensions and solutions containing the 5-ALA ester optionally together with one or more inert conventional carriers and/or diluents, e.g. with corn starch, lactose, sucrose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, stearylalcohol, carboxymethylcellulose or fatty substances such as hard fat, or suitable mixtures thereof. If the composition for use according to the invention optionally comprises one or more pharmaceutically acceptable solvents, such solvents may be a free fatty acid, a free fatty alcohol, an aqueous solution, e.g. a buffer, or water.

Preferred dosage forms for oral administration are those which are capable of releasing the 5-ALA ester as it travels down the patient's esophagus, i.e. on contact with the mucosal surface. Examples of such dosage forms include high viscosity solutions or suspensions (e.g. syrups), lozenges, pastilles, etc.

Lozenges are a suitable form for administration. On sucking, these slowly dissolve in the patient's mouth thereby releasing the 5-ALA ester down the esophagus. In one embodiment, lozenges which include components capable of slowing the passage of the active down the esophagus, thereby maximising the period of contact with the mucosal tissues, are provided. For example, these may contain a carrier, e.g. a polyhydric alcohol carrier or corn syrup and will, in general, also contain one or more thickening, lubricating and/or wetting agents.

Spraying of the compositions onto the surface of the esophagus at the desired site may also be used to administer the 5-ALA ester, either in the form of solid fine particles or in the form of tiny droplets of a solvent containing the dissolved 5-ALA ester. In one embodiment, an aerosol may deliver a fine powder comprising a 5-ALA ester which dissolves on contact with the moist mucosal surface.

The concentration of the 5-ALA ester in the compositions for use in the invention depends upon the nature of the 5-ALA ester, the mode of administration and the subject to which it is administered and may be varied or adjusted according to choice. Generally, however, concentration ranges of 0.01 to 50% by weight, such as 0.05 to 20% by weight, or 1 to 10% by weight, e.g. 1 to 5% by weight.

Pharmaceutically acceptable excipients which may be present in the compositions herein described include thickening agents. Such agents swell as they absorb liquid and thus may be used to improve the viscosity and consistency of the pharmaceutical product. Gums like gellan gum, xanthan gum, guar gum and carrageenan; or cellulose derivatives like carboxymethylcellulose and (meth)acrylates may be used as thickening agents. Other suitable thickening agents are polyacrylic acids (carbomer) or wax or waxy solids e.g. solid fatty alcohols or solid fatty acids. Other polymer materials which gel on contact with the fluids of the mucosa-lined surface of the esophagus may also be used. These include chitin, chitosan and chitosan derivatives such as chitosan salts (hydrochloride, lactate, aspartate, glutamate) and N-acetylated chitosan or N-alkylated chitosan, pectin, alginates (e.g. sodium alginate), pullulan, hyaluronic acid and derivatives thereof.

If present in the compositions, the thickening agents may conveniently be provided in such an amount that the desired viscosity is obtained. The actual amount will depend on the nature of the thickening agent and can readily be determined by those skilled in the art.

In another embodiment, the compositions may further comprise one or more bioadhesive agents, e.g. mucoadhesive agents. Bioadhesives are a class of molecules which are known to form an interaction with materials of a biological nature for an extended period of time by interfacial forces resulting in fixation to a specific biological location. Attachment is achieved by complex but non-specific mechanisms, generally via non-covalent binding such as electrostatic forces, van der Waals forces, hydrogen bonds and hydrophobic interactions. Mucoadhesive agents are a special class of bioadhesive agents which bind to mucous, particularly the glycoprotein mucin which is present in the mucous layer which lines the gastrointestinal tract primarily for protection and lubrication. Generally the attachment of the mucoadhesive to mucin is sufficiently strong so that removal occurs primarily through mucin turnover, i.e. the weaker bond is the mucin-mucin rather than the mucin-mucoadhesive bond. In addition, some agents which exhibit an affinity for mucous surfaces bind to the epithelial cells which lie beneath the mucous layer, e.g. lectins, by virtue of specific receptor-mediated interactions.

As used herein, the term “mucoadhesive agent” denotes a compound which exhibits an affinity for a mucosa surface, i.e. which adheres to that surface through the formation of bonds which are generally non-covalent in nature, whether binding occurs through interaction with the mucous and/or the underlying cells. In the context of the invention, the mucosa surface is that of the esophagus.

As will be appreciated, the bioadhesive (e.g. mucoadhesive agent) may itself comprise the carrier or excipient and thus in those cases a further carrier or excipient is optionally present.

Suitable mucoadhesive agents may be natural or synthetic compounds, polyanionic, polycationic or neutral, water-soluble or water-insoluble, but are preferably large, e.g. having a molecular weight of 500 kDa to 3000 kDa, e.g. 1000 kDa to 2000 kDa, water insoluble cross-linked, e.g. containing 0.05% to 2% cross-linker by weight of the total polymer, prior to any hydration, water-swellable polymers capable of forming hydrogen bonds. Preferably such mucoadhesive compounds have a mucoadhesive force greater than 100, especially preferably greater than 120, particularly greater than 150, expressed as a percent relative to a standard in vitro, as assessed according to the method of Smart et al., 1984, J. Pharm. Pharmacol., 36, pp 295-299. Suitable mucoadhesives are described for instance in WO 02/09690, the contents of which are incorporated herein by reference.

Some of the suitable mucoadhesives for use in the compositions herein described include, polyacrylic hydrogels, chitosan, polyvinyl alcohol, hydroxypropyl cellulose, hydroxypropyl methyl cellulose, sodium alginate, scleroglucan, xanthan gum, pectin, orabase and polygalactonic acid.

The above-mentioned mucoadhesives may be prepared using standard processes and procedures well-known in the art, although many are available commercially, e.g. from Goodrich, BDH, Hercules, Dow Chemical Co.

Mucoadhesive derivatives or salts may also be used. Appropriate salts are as described above in connection with the 5-ALA esters. Mucoadhesive derivatives include chemically modified agents which retain mucoadhesive properties. Such derivatives include those which have been modified to include the 5-ALA ester, such as ALA-methyl ester alginate or esters such as between ALA and hydroxypropyl cellulose may be formed.

The 5-ALA ester and the mucoadhesive may be combined into a composition or a product by any appropriate means. For example, depending on the mucoadhesive agent the composition may be formulated as a dry polymeric film, tablet or powder. In this case adhesion is likely to occur via hydration, as in the case of cellulose derivatives. Other formulations may be applied in a semi or fully hydrated state (e.g. hydrogels, such as poly (acrylic acid) polymers, hyaluronic acid and chitosan), e.g. by use of an aqueous solution.

When present, the mucoadhesive agent may conveniently be provided in a concentration range of 0.05 to 30%, e.g. about 1 to 25% by weight of the total weight of the pharmaceutical product.

The 5-ALA esters may be formulated and/or administered with other active components which serve to enhance the photodynamic effect, for example surface penetration assisting agents and/or chelating agents. Dependent on the nature of the composition, the 5-ALA ester may be co-administered with such other optional agents, for example in a single composition or, alternatively, they may be administered separately (e.g. sequentially). In some cases it may be beneficial to pre-treat the surface of the esophagus with a surface penetration assisting agent in a separate step prior to administration of the active component. When a surface penetration assisting agent is used in a pre-treatment step, this may be used at higher concentrations, e.g. up to 100% by weight. If such a pre-treatment step is used, the active component may subsequently be administered up to several hours following pre-treatment, e.g. at an interval of 5 to 60 minutes following pre-treatment.

Products and kits which comprise a composition as herein described and, optionally, a surface penetration assisting agent and/or a chelating agent, as a combined preparation for simultaneous, separate or sequential use in a method of photodynamic diagnosis of an abnormality of the epithelial lining of the esophagus form further aspects of the invention.

Alternatively viewed, this aspect of the invention provides a kit for use in a method of photodynamic diagnosis of an abnormality of the epithelial lining of the esophagus as herein described, said kit comprising:
(a) a first container containing a 5-ALA ester or a pharmaceutically acceptable salt thereof, or a pharmaceutical composition as herein described;
(b) a second container containing at least one surface penetration assisting agent; and optionally
(c) one or more chelating agents contained either within said first container or in an optional third container.

The compositions may, for example, further comprise one or more surface penetration enhancing agents. Such agents may have a beneficial effect in enhancing the photosensitizing effect of the 5-ALA ester present in the compositions. Preferred surface penetration enhancing agents are chelators (e.g. EDTA), surfactants (e.g. sodium dodecyl sulfate), non-surfactants, bile salts (sodium deoxycholate), fatty alcohols e.g. oleylalcohol, fatty acids e.g. oleic acid and esters of fatty acids and alcohol, e.g. isopropylmyristate. Examples of appropriate surface penetrating assisting agents include isopropanol, HPE-101 (available from Hisamitsu), DMSO and other dialkylsulphoxides, in particular n-decylmethyl-sulphoxide (NDMS), dimethylsulphacetamide, dimethylformamide (DMFA), dimethylacetamide, glycols, various pyrrolidone derivatives (Woodford et al., J. Toxicol. Cut. & Ocular Toxicology, 1986, 5: 167-177), and Azone® (Stoughton et al., Drug Dev. Ind. Pharm. 1983, 9: 725-744), or mixtures thereof. DMSO is, however, preferred due to its anti-inflammatory activities and its stimulatory effect on the activity of the enzymes ALA-synthase and ALA-dehydrogenase (the enzymes which, respectively, form and condense ALA to porphobilinogen) thereby enhancing the formation of the active form, Pp IX.

When present, the surface penetration enhancing agent may conveniently be provided in a concentration range of 0.2 to 20% by weight of the total weight of the composition, e.g. about 1 to 15% or 0.5 to 10% by weight of the total weight of the composition.

The composition may further comprise one or more chelating agents. Such agents may also have a beneficial effect in enhancing the photosensitizing effect of the 5-ALA ester present in the composition. Chelating agents may, for example, be included in order to enhance the accumulation of PpIX since the chelation of iron by the chelating agent promotes its incorporation into PpIX to form haem by the action of the enzyme ferrochelatase, thereby leading to a build up of PpIX. The photosensitizing effect is therefore enhanced.

Suitable chelating agents are aminopolycarboxylic acids and any of the chelants described in the literature for metal detoxification or for the chelation of paramagnetic metal ions in magnetic resonance imaging contrast agents. Particular mention may be made of EDTA, CDTA (cyclohexane triamine tetraacetic acid), DTPA and DOTA and well known derivatives and analogues thereof. EDTA and DTPA are particularly preferred. Other suitable chelating agents are desferrioxamine and siderophores and they may be used alone or in conjunction with aminopolycarboxylic acid chelating agents such as EDTA. Some of these chelating agents do also exhibit surface penetration assisting agent properties, e.g. EDTA.

Where present, the chelating agent may conveniently be used at a concentration of 0.01 to 12%, e.g. 0.1 to 5% by weight based on the total weight of the composition.

The compositions for use in the invention may further comprise one or more pharmaceutically acceptable excipients which are different from the aforementioned excipients. Such excipients are for instance surfactants, emulsifiers, such as non-ionic or cationic emulsifiers, fillers, binders, spreading agents, solubilizing and/or stabilizing agents (e.g. cyclodextrins (CD) α, β, γ and HP-β cyclodextrin), lubricating agents, flavours (e.g. sweetening agents), odor enhancers, solvents, suspending agents, wetting agents or preservatives. The skilled man will be able to select suitable excipients based on their purpose. Common excipients that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe fur Pharmazie, Kosmetik and angrenzende Gebiete (Edition Cantor Aulendorf, 1989)).

All of the above-mentioned pharmaceutically acceptable excipients are well known in the art and are commercially available from various manufacturers.

After administration of the composition to the surface of the esophagus, the desired area for examination is exposed to light to achieve the desired photoactivation and photodynamic diagnosis. The length of time period between administration and exposure to light will depend on the nature of the 5-ALA ester, the nature of the composition and its mode of administration. Generally, it is necessary that the 5-ALA ester within said composition is sufficiently released to be taken up by the cells of the tissue to be diagnosed, converted into a photosensitizer and achieves an effective tissue concentration at the site of diagnosis prior to photoactivation.

Typically, the incubation time will be up to 10 hours, for example about 10 minutes to 10 hours, e.g. 30 minutes to 7 hours, or 1 hour to 5 hours. Direct local administration will result in shorter waiting times between administration and activation, for example, 10 minutes to 3 hours, e.g. 15 minutes to about 2 hours or 30 minutes to 1 hour. Where systemic uptake is required following oral administration, the waiting time will be longer, for example between 1 and 6 hours, e.g. 2 to 5 hours or 3 to 4 hours. Optimum waiting times to maximise the concentration of the photosensitizer at the target site may readily be determined.

The irradiation will generally be applied for a short time with a high light intensity, i.e. a high fluence rate or for a longer time with a low light intensity, i.e. low fluence rate.

The wavelength of the light used for irradiation may be selected to achieve the desired photodynamic effect. Blue light having a wavelength typically ranging from 360 to 450 nm will generally be used, giving rise to fluorescence which is measured in the red region (e.g. 550 to 750 nm).

Methods for irradiation, e.g. by lamps or lasers, are well known in the art. The irradiation will in general be applied at a dose level of 10 to 100 Joules/cm² with an intensity of 20-200 mW/cm² when a laser is used, or a dose of 10-100 J/cm² with an intensity of 50-150 mW/cm² when a lamp is applied. Conveniently, a fibre optic carrying a laser light may be fed through an endoscope to accomplish exposure to irradiation. Exposure may be adjusted, as required, by varying the time of exposure and/or the light intensity.

Fluorescence from the excited photosensitizer may be detected using any conventional fluorescence detector. The emitted fluorescence (e.g. at about 635 nm) is used to selectively detect abnormal cells or tissues, e.g. affected cancerous tissue or pre-cancerous tissue (e.g. dysplasia) or non-cancerous conditions (e.g. metaplasia). In a preferred embodiment of the invention, the fluorescence detected may be used to produce an image of the desired area or areas of the esophagus. Different levels of fluorescence intensity may then be used to identify the different types of cells which may be present. Those areas in which pre-cancerous (e.g. dysplastic) or cancerous cells are present give rise to a higher fluorescence intensity, i.e. a positive fluorescence contrast, compared to normal squamous epithelial tissue. Those areas in which metaplastic cells are present give rise to a lower fluorescence intensity, i.e. a negative fluorescence contrast, compared to normal cells. Thus, positive and negative contrast in a fluorescence intensity image may be used to map the different areas of the esophagus.

In a further aspect the invention thus provides a method of in vivo imaging of a portion of the epithelial lining of the esophagus, said method comprising the following steps:
(a) administering to a subject, e.g. a human or non-human animal, a 5-ALA ester or pharmaceutically acceptable salt thereof;
(b) if necessary, waiting for a time period necessary for the 5-ALA ester or pharmaceutically acceptable salt thereof to be converted into a photosensitizer and achieve an effective tissue concentration at the desired target site in the esophagus;
(c) exposing the epithelial lining of the esophagus to light whereby to photoactive the photosensitizer;
(d) detecting a fluorescence intensity indicative of an abnormality of the epithelial lining of the esophagus; and
(e) converting the detected fluorescence into an image of an area of interest in the epithelial lining of the esophagus.

In the imaging method, differences in fluorescence intensity from different tissue types present at the target site may be detected, these differences in fluorescence intensity being indicative of the presence or absence of an abnormality.

In a preferred embodiment of this aspect of the invention, the method of imaging may be performed on a subject pre-administered with said 5-ALA ester or pharmaceutically acceptable salt thereof.

In a preferred embodiment of the methods herein described, detection of a low fluorescence intensity is indicative of the presence of a metaplastic tissue or a normal tissue, whereby the fluorescence of the metaplastic tissue is lower compared to the fluorescence of the normal tissue. In another preferred embodiment, detection of a high fluorescence intensity is indicative of a dysplastic, neoplastic or cancerous tissue, whereby the fluorescence of the dysplastic and neoplastic tissue is lower compared to the fluorescence of the cancerous tissue.

Suitable endoscopes are known in the art and may be adapted to allow emission of blue light (for excitation purposes) in addition to white light, e.g. by being equipped with an internal filter assembly which passes primarily blue light. A foot pedal may be used to allow convenient switching between white and blue light. The light source may be a laser or a lamp. To visualize fluorescence, the endoscope may be equipped with an integrated filter which blocks most of the reflected blue light. A camera, such as a modified color charge-coupled device (CCD) camera, may be used to capture images of the esophagus and a standard color monitor may be used to display images of the esophagus. Irradiation is preferably performed for 2 to 60 minutes, e.g. from 5 to 30 minutes. A single irradiation may be used or alternatively a light split dose in which the light dose is delivered in a number of fractions, e.g. a few minutes to a few hours between irradiations, may be used. Multiple irradiations may also be applied. The area of examination may further be inspected by use of white light, e.g. before, during or after irradiation with blue light. Cancerous tissue or pre-cancerous tissue identified due to its fluorescence may be removed during irradiation or in white light.

Following identification of the abnormality using any of the methods herein described, this may then be treated through alternative therapeutic techniques, e.g. by surgical or chemical treatment. Examples of current treatments include surgical treatment, endoscopic ablation therapy, chemical ablation (e.g. by PDT), thermal ablation or mechanical ablation. In one embodiment of the invention, further application of the 5-ALA ester compound at the site of interest may be carried out in order to effect PDT. It will be appreciated that PDT may require higher concentrations of 5-ALA esters for destruction of the abnormal cells or tissues than used in the diagnostic methods. Generally, concentration ranges of up to 50% by weight, preferably 10 to 50% by weight, e.g. 15 to 30% by weight, are suitable. Methods and modes of administration have been considered before with regard to the diagnostic uses and are applicable also to therapeutic methods.

Although it is anticipated that the diagnostic techniques herein described will primarily be carried out in vivo, these may also be carried out in vitro in order to examine cells or tissues which have been removed from the esophagus by biopsy.

In a further aspect the invention thus provides a method of in vitro diagnosis of an abnormality of the epithelial lining of the esophagus by assaying a sample of body tissue from the esophageal lining, said method comprising at least the following steps:
(i) admixing said body tissue with a 5-ALA ester or pharmaceutically acceptable salt thereof;
(ii) exposing said mixture to light;
(iii) ascertaining the level of fluorescence; and
(iv) comparing the level of fluorescence to control levels.

In another aspect, the present invention relates to esophageal stents for use in the methods of photodynamic diagnosis disclosed herein, more specifically an esophageal stent which is coated with a 5-ALA ester or a pharmaceutically acceptable salt thereof.

Stents for use in delivering therapeutic agents, including photosensitive agents, to a body lumen have been described previously, however these have been developed for very different applications to the present invention. Primarily, these are described for use in treating conditions within the vascular system where the stent serves to maintain the desired shape of the vessel, e.g. by opening or maintaining a passageway for the flow of blood. In such cases, the therapeutic agent is chosen for its ability to prevent or reduce any constrictive intraluminal disease such as restenosis.

For example, WO 2007/030478 describes a stent coated with an energy-activatable agent for long-term implantation into vessels to prevent obstruction of the vessel (e.g. by breaking down atherosclerotic plaques in arteries), the release of the active agent being controlled by a light stimulus which may be delivered to the patient after an extended time period.

Similarly, WO 94/15583 describes a slow-release drug-eluting stent for treatment of atherosclerosis, in particular restenosis of blood vessels following treatment of atherosclerotic plaques, by destroying the proliferating smooth muscle cells lining the vessel using a photosensitive agent and so preventing further obstruction. The purpose of this device is to maintain patency of vessels both by its physical presence in the vessel and by delivering active agents to breakdown proliferating cardiovascular tissue and thereby prevent flow obstruction. Such a device is not suitable for use in the esophagus, and does not address the problem of providing an alternative method for the controlled delivery of photosensitive agents for use in the diagnosis and treatment of esophageal abnormalities such as Barrett's esophagus.

The esophageal stent for use in the method of the invention comprises a generally cylindrical stent body having a surface coating comprising a 5-ALA ester or a pharmaceutically acceptable salt thereof (also referred to herein as the “active agent”).

Stents, including those suitable for use in the esophagus, are generally known and used in the art and may take a variety of forms. Any known type of stent which is suitable for deployment in the esophagus may be employed in the invention. The underlying structure (i.e. framework) of the stent body is not particularly limiting provided that this is capable of delivery to the esophagus and can be held in place (e.g. following radial expansion) to allow for contact with the surface tissue of the esophagus and delivery of the active agent.

The stent may be of the self-expandable type or the balloon-expandable type and will typically comprise an open framework onto which the active agent can be applied. The framework will generally be such that the stent can be delivered in a collapsed state (e.g. crimped onto a catheter) to enable this to pass safely down the esophagus before being expanded at the deployment site. Once in position, the stent can then be expanded, e.g. by inflation of a dilation balloon.

The stent body can be made of any material which is acceptable for introduction into the body. For example, this may be made of any bio-compatible material having the necessary physical characteristics for the chosen design of stent. Suitable stent materials are those which will not irritate the esophageal wall following deployment or cause any adverse reaction and include metals and polymers.

Examples of suitable metals include stainless steel, tantalum, cobalt alloys (e.g. cobalt-chromium alloys), platinum alloys (e.g. platinum-iridium alloys), and nickel alloys (e.g. nickel-titanium alloys).

Polymeric stents may be made from biostable or biodegradable polymer materials. Those made from biostable materials will generally be removed once the active agent has been delivered to the esophageal wall or, dependent on their light transmission characteristics, after PDD has been carried out. Biodegradable polymers are those which degrade or dissolve in the body. These have the advantage that they may not need to be removed from the esophagus following deployment, especially if these are transparent to light and so may be left in place during the PDD procedure. Depending on the nature of the polymer material, in some cases these may degrade before photoactivation is carried out.

Suitable biodegradable polymeric materials may be selected from the group consisting of poly-lactic acids, poly-glycolic acids, collagen, polycaprolactone, hyaluronic acid, polydioxanone, polycaprolactone, polygluconate, poly-lactic acid-polyethylene oxide copolymers, poly(hydroxybutyrate), polyanhydrides, polyphosphoesters, poly(amino acids), and poly(alpha-hydroxy acids). The nature of the biodegradable polymer may be chosen depending on the desired absorption rate.

Biostable polymers may also be used to form the stent body. Such materials include silicones, poly(ethylene terephthalate) and polyacetals.

Polymer-coated metals may also be used to form the stent body. These may comprise any combination of the polymer materials and metals hereinbefore described.

The stent body will typically comprise a framework or mesh made from a plurality of filaments or ‘struts’ which may be interwoven or inter-linked. Often the filaments will form a zig-zag or sinusoidal wave structure which enables the stent to be radially expanded from a contracted state. Depending on the precise geometry of the stent filaments, the stent coating which carries the active agent will generally cover the entire stent surface, i.e. both the inner and outer surfaces. However, in some cases, the stent coating will only be applied to the outer (i.e. tissue-contacting) surface. The coating may consist of a single coating or multiple coatings. Where multiple coatings are present, one or more of the individual layers may contain the active agent, preferably all layers. In some cases, it may, however, be desirable to apply an outer protective layer which need not necessarily contain any active agent.

The size of the stent may readily be selected according to need and will depend on factors such as the age and size of the patient to be treated, the extent of the abnormality to be treated or diagnosed, etc. The stent diameter should be such that, in use (i.e. in the expanded state), its outer surface contacts the esophagus wall such that the active agent can be delivered by contact transfer. The length of the stent may be chosen according to the extent of the area or areas within the esophagus to be treated. For example, a longer stent extending up to 25 or 30 cm may be used in a method of treatment or diagnosis to be performed on the entire length of the esophagus. Shorter stents may be used to treat a more localised area or areas.

The thickness of the stent coating will be dependent on factors such as the desired concentration of the active agent, the nature and amount of any other excipients which may be present in the coating, the presence of multiple coating layers (e.g. whether there is any base coat and/or top coat present), etc. Suitable thicknesses can readily be determined by those skilled in the art. Typically, these will be of the order of several micrometres, e.g. from about 3 to about 20 micrometres, or from about 5 to about 10 micrometres.

In cases where it is desirable that the stent should be biodegradable, the thickness of the stent body and coating should be selected accordingly. Suitable thicknesses may readily be selected such that the stent degrades entirely in vivo over a time period ranging from 2 to 24 hours.

Suitable coating materials and methods for application of these to the stent body, in particular to its tissue-contacting surface, are generally known in the art, e.g. in WO 2012/004399 to Photocure ASA, the contents of which are hereby incorporated by reference. These should be capable of securing an effective amount of the desired active agent onto the body of the stent (the active should be present in a therapeutically or diagnostically effective amount); and able to keep this in place during delivery and expansion of the stent in vivo. In certain cases, the coating may be selected such that this controls the rate of delivery of the active agent from the stent to the esophageal wall.

Methods suitable for use in preparing the coated stents herein described may comprise the following steps:
(a) providing a generally cylindrical stent body; and
(b) applying to said body a coating comprising the 5-ALA ester or a pharmaceutically acceptable salt thereof.

In one embodiment, the coating may be provided in the form of a powder, i.e. a dry, bulk solid composed of a large number of very fine particles, more preferably in the form of a compressed powder, i.e. having lost its ability to flow. In another embodiment, the dry pharmaceutical composition is in the form of a cake, i.e. a dry bulk solid formed into a small block. In a preferred embodiment, the coating is in the form of a film, i.e. one or more (thin) layers of dry/dried material, preferably a relatively homogeneous film which covers substantially the whole surface of the stent or substantially the outer surface of the stent. In a further preferred embodiment, this film is well attached and stays well attached to the stent surface, i.e. is relatively stable under mechanical stress which may occur during transport and shipment and during deployment in vivo.

In one embodiment, the coating may be obtained as a film by film coating processes known in the art, preferably by dip-coating or spray-coating. Such methods are discussed in detail in WO 2012/004399, the contents of which are incorporated herein by reference.

In a preferred embodiment, one or more polymers and optionally other pharmaceutically acceptable excipients are present in the coatings herein described. Preferred one or more polymers are polymers which have good film-forming properties and/or good gel forming properties. Preferred other pharmaceutically acceptable excipients are selected from one or more of the following compounds: plasticizers, coloring agents and thickening agents. Other pharmaceutically acceptable excipients which may be present in the coating are disintegrants, mucoadhesive agents, surface penetration enhancing agents and chelating agents.

If one or more polymers and/or pharmaceutically acceptable excipients are present in the coating, the active agent may be present in the range of 0.25 to 50%, for example 0.5 to 30%, such as 0.5 to 15% or 1 to 10% or 1 to 7% by weight of the total weight of the coating. Alternatively, if only one or more polymers are additionally present in the dry pharmaceutical composition, the active agent may be present in the range of 50 to 99%, for example 60 to 91% or 75 to 90% by weight of the total weight of the coating. By having a high amount of active agent compared to the amount of the one or more polymer, the liquid which is used to deposit the composition is less viscous and thus easier to handle and process. In another embodiment if one or more polymers and pharmaceutically acceptable excipients selected from plasticizers are present in the coating, the active agent may be present in the range of 15 to 85%, for example 20 to 80% such as 25 to 78% or 26 to 60% by weight of the total weight of the coating.

All one or more polymers and pharmaceutically acceptable excipients should be non-toxic, non-irritant and devoid of leachable impurities. They should be inert towards the active agent, i.e. should not promote its degradation. One or more of each pharmaceutically acceptable excipient compound may be used, e.g. one or more plasticizers, one or more coloring agents, etc.

The one or more polymers for use in the coatings can be natural, semi-natural, i.e. derivatives of natural polymers which are obtained by a chemical reaction, or synthetic polymers; they may be homopolymers or copolymers.

Preferably, polymers are used which have good film-forming properties, i.e. which form—together with the active agent—a film when deposited on the stent surface. A preferred group of such polymers are starch, cellulose and derivatives of starch and cellulose. Preferred starch derivatives are starch acetate and carboxymethyl starches, preferably with an amylose content of at least 18% by weight. One preferred cellulose is microcrystalline cellulose. Other preferred cellulose derivatives are cellulose ethers such as methylcellulose, ethylcellulose, hydroxyethylcellulose, hydroxypropylcellulose, hydroxypropylmethylcellulose, hydroxypropylethyl-cellulose and carboxymethylcellulose. Such polymers may be used in combination with other polymers, e.g. ethylcellulose with hydroxypropylmethylcellulose. Other preferred cellulose derivatives are cellulose acetate phthalate and nitrocellulose. Further preferred polymers are rosin and rosin esters. Another preferred group of polymers are (meth) acrylate polymers and copolymers. The use of “meth” as a prefix in parenthesis indicates, in accordance with common practice, that the polymer molecule is derived from monomers having the carbon atom skeleton of either or both of acrylic acid and methacrylic acid. Such polymers and copolymers are e.g. based on methylmethacrylate, ethylacrylate, methacrylic acid and trimethylammonioethylmethacrylate chloride, e.g. anionic and cationic polymers of methacrylic acid, copolymers of methacrylates, copolymers of acrylates and methacrylates, copolymers of ethylacrylates and methylmethacrylates. Other preferred polymers are polyvinyl acetate phthalate. In a more preferred embodiment, cellulose and cellulose derivatives, especially cellulose ethers, are used as one or more polymers in the coatings herein described.

In another embodiment, polymers with good gel-forming properties may be used, i.e. which form—together with the active agent—gels on contact with water and fluids of mucosa lined surfaces. Preferred such polymers are gums, preferably gellan gum, xanthan gum and carrageenan. Other preferred polymers are chitin, chitosan and chitosan derivatives such as chitosan salts (hydrochloride, lactate, aspartate, glutamate) and N-acetylated chitosan or N-alkylated chitosan. Yet other preferred polymers are pectin, alginates, e.g. sodium alginate, pullulan, hyaluronic acid and derivatives thereof.

Preferably, in yet another embodiment, polymers with good film-forming properties and good gel-forming properties are used, e.g. cellulose ethers like methylcellulose, ethylcellulose, gellan gum, chitosan and chitosan derivatives, pullulan, alginates, hyaluronic acid, derivatives of hyaluronic acid or carrageenan. Preferred such polymers are cellulose ethers like methylcellulose, ethylcellulose, hydroxyethylcellulose, hydroxypropylcellulose, hydroxypropylmethylcellulose, hydroxypropylethylcellulose and carboxymethylcellulose and chitosan and chitosan derivatives.

Polymers with good film-forming properties are preferably used if the coating should form a film.

The polymers may be water soluble or insoluble in water. In a preferred embodiment, water soluble polymers are used.

If present in the coating, the one or more polymers may conveniently be provided in a concentration range of 50 to 99.75%, for example 70 to 99.5%, e.g. 85 to 99.5%, or 90 to 99% or 93 to 99% by weight of the total weight of the coating. Alternatively, if only one or more polymers are additionally present in the coating, the one or more polymers may be present in the range of 1 to 50%, for example 9 to 40% or 10 to 25% by weight of the total weight of the coating. By having a high amount of active agent compared to the amount of the one or more polymer, the liquid which is used to deposit the composition is less viscous and thus easier to handle and process. In another embodiment if one or more polymers and pharmaceutically acceptable excipients selected from plasticizers are present in the coating, the one or more polymers may be present in the range of 20 to 65%, for example 25 to 62% such as 30 to 55% or 40 to 54% by weight of the total weight of the coating.

Other pharmaceutically acceptable excipients which may be present in the coating are plasticizers. In general their use is to reduce the glass transition temperature of a polymer making it more elastic and deformable, i.e. flexible. Hence they may be present in the coating if one or more polymers are present, preferably if one or more film-forming polymers are present. Plasticizers are preferably chosen in such a way that they work well with the given polymer(s). In one embodiment, suitable plasticizers are acting as a good solvent for the polymer(s) in question. In another embodiment, if water soluble polymers are used, the plasticizer is preferably a water miscible compound. Suitable plasticizers are low molecular weight polyethylene glycols, phthalate derivatives like dimethyl, diethyl and dibutyl phthalate, citrate esters such as triethyl, tributyl and acetyl citrate, dibutyl sebacate, camphor, triacetin, oils and glycerides such as castor oil, acetylated monoglycerides and fractionated coconut oil. Glycerol and propylene glycol are also common plasticizers, they should however not be used if the coating contains as an active ingredient lower alkyl ALA esters or salts thereof, such as C₁-C₈-alkyl ALA esters since they may promote degradation of such active ingredients.

If present in the coating, the plasticizers may conveniently be provided in a concentration range of 1 to 30%, for example 5 to 20% or 7 to 15% by weight of the total polymer weight. Alternatively, the plasticizers may be provided in a higher concentration range, for instance in a concentration range of 10 to 175%, or 35 to 150% or 37 to 80% by weight of the total polymer weight.

Other pharmaceutically acceptable excipients which may be present in the coating are coloring agents, such as synthetic dyes or pigments, e.g. titanium dioxide or yellow iron oxide. Pigments usually decrease the permeability of the coating to water vapor and oxygen and may thus increase its shelf life. Further, they contribute to the total solids of the liquid used to obtain the coating without significantly contributing to its viscosity. Thus faster processing time by virtue of more rapid drying is possible, which is particularly significant for aqueous based liquids used in spray-coating. If present in the coating, the coloring agents may conveniently be provided in a concentration range of 0.1 to 20%, for example 0.5 to 10% or 1 to 5% by weight of the coating.

Other pharmaceutically acceptable excipients which may be present in the coating are thickening agents. Such agents swell as they absorb liquid and thus may be used to improve the viscosity and consistency of the liquid which is used to obtain the coating. Preferably, thickening agents are used in liquids which are employed in dip-coating. The choice of thickening agents is dependent on whether the liquid is an aqueous or aqueous based liquid or whether non-aqueous solvents are used to form the liquid. Some of the afore-mentioned polymers have thickening properties, e.g. gums like guar gum, cellulose derivatives like carboxymethylcellulose and (meth)acrylates. Other suitable thickening agents are polyacrylic acids (carbomer) or wax or a waxy solids e.g. solid fatty alcohols or solid fatty acids. If present in the coating, the thickening agents may conveniently be provided in such an amount that the desired viscosity of the liquid described above is obtained. The actual amount will depend on the one or more solvent said liquid comprises and the nature of the thickening agent.

Other pharmaceutically acceptable excipients which may be present in the coating are disintegrants. Generally, disintegrants aid in the break up of the coating when it is put into a moist environment. Some of the aforementioned polymers do exhibit disintegrant properties, e.g. certain celluloses, starch and derivatives thereof. If these polymers are present, there may not be a need or desire to add any further disintegrants. More effective disintegrants are referred to as superdisintegrants. Those include for instance alginic acid, croscarmellose, crospovidone and sodium starch glycolate. Such compounds swell when they come in contact with fluids but they do not form a gel which would decrease their disintegration properties. If present in the coating, the disintegrants may conveniently be provided in a concentration range of 0.1 to 10% by weight of the total weight of the coating, for example 0.25 to 5% or 0.5 to 4% by weight.

The coatings may further comprise one or more mucoadhesive agents. The term “mucoadhesive agent” denotes a compound which exhibits an affinity for a mucosa surface, i.e. which adheres to that surface through the formation of bonds which are generally non-covalent in nature, whether binding occurs through interaction with the mucous and/or the underlying cells.

Suitable mucoadhesive agents may be natural or synthetic compounds, polyanionic, polycationic or neutral, water-soluble or water-insoluble, but are preferably large, e.g. having a molecular weight of 500 kDa to 3000 kDa, e.g. 1000 kDa to 2000 kDa, water-insoluble cross-linked, e.g. containing 0.05% to 2% cross-linker by weight of the total polymer, prior to any hydration, water-swellable polymers capable of forming hydrogen bonds. Preferably such mucoadhesive compounds have a mucoadhesive force greater than 100, especially preferably greater than 120, particularly greater than 150, expressed as a percent relative to a standard in vitro, as assessed according to the method of Smart et al., 1984, J. Pharm. Pharmacol., 36, pp 295-299.

Some of the afore-mentioned polymers exhibit mucoadhesive properties, for instance gums like guar gum, chitosan and chitosan derivatives, pullulan, sodium alginate or hyaluronic acid. If these polymers are present, there may not be a need or desire to add any further mucoadhesive agents.

Suitable mucoadhesive agents are selected from polysaccharides, preferably dextran, pectin, amylopectin or agar; gums, preferably guar gum or locust bean gum; salts of alginic acid, e.g. magnesium alginate; poly(acrylic acid) and crosslinked or non-crosslinked copolymers of poly(acrylic acid) and derivatives of poly(acrylic acid) such as salts and esters like for instance carbomer (carbopol).

When present, the mucoadhesive agent may conveniently be provided in a concentration range of 0.05 to 30%, e.g. about 1 to 25% by weight of the total weight of the coating.

The coating may further comprise one or more surface penetration enhancing agents. Such agents may have a beneficial effect in enhancing the photosensitizing effect of the active agent, e.g. of 5-ALA, the derivative of 5-ALA or the precursor of 5-ALA present in the coating. Preferred surface penetration enhancing agents are chelators (e.g. EDTA), surfactants (e.g. sodium dodecyl sulfate), non-surfactants, bile salts (sodium deoxycholate), fatty alcohols e.g. oleylalcohol, fatty acids, e.g. oleic acid and esters of fatty acids and alcohol, e.g. isopropylmyristate. When present, the surface penetration enhancing agent may conveniently be provided in a concentration range of 0.2 to 20% by weight of the total weight of the coating, e.g. about 1 to 15% or 0.5 to 10% by weight of the total weight of the coating.

The coating may further comprise one or more chelating agents. Such agents may also have a beneficial effect in enhancing the photosensitizing effect of the active agent. Their ability to enhance the photosensitizing effect of the active agent means that a lower amount of the active agent needs to be taken up by the cells in order to achieve the desired photodynamic effect. This has the advantage that less time is generally needed for the cells to take up an effective amount of the photosensitizing agent or precursor thereof.

Chelating agents may, for example, be included in order to enhance the accumulation of PpIX since the chelation of iron by the chelating agent prhomogeneousts its incorporation into PpIX to form haem by the action of the enzyme ferrochelatase, thereby leading to a build up of PpIX. The photosensitizing effect is therefore enhanced. Suitable chelating agents are aminopolycarboxylic acids and any of the chelants described in the literature for metal detoxification or for the chelation of paramagnetic metal ions in magnetic resonance imaging contrast agents. Particular mention may be made of EDTA, CDTA (cyclohexane triamine tetraacetic acid), DTPA and DOTA and well known derivatives and analogues thereof. EDTA and DTPA are particularly preferred. Other suitable chelating agents are desferrioxamine and siderophores and they may be used alone or in conjunction with aminopolycarboxylic acid chelating agents such as EDTA. Some of these chelating agents do also exhibit surface penetration assisting agent properties, e.g. EDTA. Preferred chelators are iron-chelators such as CP 94 (1,2-diethyl-3-hydroxypyridin-4-one) as described in Bech et al., Journal of Photochemistry and Photobiology B: Biology 41 (1997), 136-144, or Deferasirox such as described in U.S. Pat. No. 6,596,750. Where present, the chelating agent may conveniently be used at a concentration of 0.01 to 12%, e.g. 0.1 to 5% by weight based on the total weight of the coating.

The coating may further comprise one or more pharmaceutically acceptable excipients which are different from the afore-mentioned excipients. Such excipients are for instance surfactants, emulsifiers, preferably non-ionic or cationic emulsifiers, fillers, binders, spreading agents, stabilizing agents or preservatives. The skilled man will be able to select suitable excipients based on their purpose. Common excipients that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe fur Pharmazie, Kosmetik and angrenzende Gebiete (Edition Cantor Aulendorf, 1989)).

All of the above-mentioned pharmaceutically acceptable excipients are well known in the art and are commercially available from various manufacturers.

Following preparation, the coated stent should be covered or sealed such that this is retained in a moisture-free environment prior to use, for example in an airtight/moisture tight bag.

The stent according to the invention can be used to deliver the active agent to the esophageal wall by introducing the stent down the esophagus and, if necessary, radially expanding this into contact with the desired portion of the esophageal wall. Esophageal delivery may be achieved using known techniques such as by a catheter or using other stent expanding devices. Radial expansion may be achieved by balloon expansion of the stent (e.g. where a balloon catheter is used) or by self-expansion. When the stent is balloon-expandable, this will generally be formed in the expanded state, crimped onto a balloon dilation catheter or other stent expanding device for delivery. At the desired site within the esophagus, the stent is expanded into place, e.g. by the radial expansion of the balloon.

The active agent is released from the stent coating in a moist environment, i.e. upon contact with the mucosa-lined surface of the esophagus. In order to achieve a full release of the active agent, the one or more polymers or other pharmaceutically acceptable excipients need to be chosen in such a way that they are either dissolved upon contact with the mucosa-lined surfaces of the esophagus or at least disintegrate to allow release of the active ingredient. The excipients, especially any polymers present, thus need to be dissolved or disintegrated at the given pH on said mucosa-lined surface.

In a further aspect the invention thus provides the use of an esophageal stent in any of the methods herein described, wherein the stent has a coating capable of releasing a 5-ALA ester or a pharmaceutically acceptable salt thereof, e.g. 5-ALA hexyl ester.

The release profile (immediate/quick, sustained and delayed release) and the residence time of the coating at the target area of the esophagus in which treatment or diagnosis is to be performed, can be influenced by the choice of the polymer as well. A quick release of the active agent may be preferred if a comparably high concentration of the active agent at the site of treatment is desired. A delayed release of the active ingredient may be preferred if a low concentration of the active agent at the site of treatment is desired. For PDD, an immediate release of the active is generally preferred. Preferred are coatings formulated for release of the active ingredient within a period of from 15 minutes to 1 hour, preferably 20 to 45 minutes, following deployment of the stent.

Following delivery of the active agent to the target site, the stent may be removed prior to carrying out PDD. Depending on the nature of the stent body, however, this may not be necessary and indeed, in some cases, it can be desirable to leave the stent in situ whilst carrying out photoactivation since this has the benefit of holding open the esophagus such that the light dose necessary to activate the photosensitizer can readily reach the esophageal surface. Polymeric stents, for example, may be transparent to light or at least sufficiently transparent that they permit an effective light dose to reach the target site and to permit the resulting fluorescence to be observed. Such “transparent” stents therefore need not be removed before PDD. After PDD has been carried out these may either be removed or, in the case where these are biodegradable, may be left at the site. Metallic stents are not transparent to light and so will typically be removed before carrying out photoactivation.

In a further aspect the invention thus provides a method of photodynamic diagnosis of an abnormality of the epithelial lining of the esophagus, said method comprising the following steps:
(a) delivering to the esophagus of a subject, e.g. a human or non-human animal, an esophageal stent as herein described;
(b) if necessary, waiting for a time period necessary for the 5-ALA ester, or pharmaceutically acceptable salt thereof, to be converted into a photosensitizer and achieve an effective tissue concentration at the desired target site in the esophagus;
(c) optionally removing the stent;
(d) exposing the epithelial lining of the esophagus to light whereby to photoactivate the photosensitizer; and
(e) detecting a fluorescence intensity indicative of said abnormality.

Another aspect of the invention is a 5-ALA ester, or a pharmaceutically acceptable salt thereof, for use in a method of photodynamic diagnosis of an abnormality of the epithelial lining of the esophagus, said method comprising the following steps:
(a) delivering to the esophagus of a subject, e.g. a human or non-human animal, an esophageal stent as herein described;
(b) if necessary, waiting for a time period necessary for the 5-ALA ester or pharmaceutically acceptable salt thereof to be converted into a photosensitizer and achieve an effective tissue concentration at the desired target site in the esophagus;
(c) optionally removing the stent;
(d) exposing the epithelial lining of the esophagus to light whereby to photoactivate the photosensitizer; and
(e) detecting a fluorescence intensity indicative of said abnormality.

The invention is illustrated by the following non-limiting example and with reference to the accompanying figures in which:

FIG. 1 is a graph showing normalized fluorescence of PpIX in the esophagus following administration of 5-ALA and 5-ALA hexyl ester; and

FIG. 2 is a graph showing the intensity of PpIX fluorescence in the Barrett's (metaplastic) and normal mucosa following administration of 5-ALA hexyl ester.

### EXAMPLE 1

### Comparative Study of the Fluorescence Induced by 5-ALA and 5-ALA Hexyl Ester in the Mucosae of the Human Esophagus Presenting a Barrett Type of Mucosae

Methods and Materials:

Spectrofluorimetry (LIFS) measurements (fluorescence intensity of PpIX in the esophagus) were carried out on 8 patients suffering from Barrett's esophagus. The measurements were carried out after oral administration of 5-ALA (ALA) or local instillation of 5-ALA hexyl ester (HAL) and the fluorescence intensity of normal and Barrett's tissue was compared.

LIFS was carried out using the Karl Storz D-light system. Abnormal cells could be clearly delineated against normal tissue due to the porphyrin fluorescence resulting from an accumulation of the ALA or HAL in the abnormal tissue. For this purpose, blue light of a specific spectral composition was introduced into the body via an endoscopic light guide system.
Patient group 1: 5 subjects each received 20 mg/kg body weight ALA orally, 4-5 hours before endoscopy.
Patient group 2: 3 subjects each received a 8 mM solution of HAL in an aqueous buffer by local instillation (100 mg in 50 mL, i.e. 0.2%), 2 hours before endoscopy.

Results:

Normalised fluorescence intensity of PpIX in the esophagus is shown in FIG. 1 for both patient groups. The large standard deviation that can be seen in this figure is due to the interpatient fluctuation of the fluorescence intensity. The intensity of fluorescence induced by HAL presents a profile between the normal epithelium and the Barrett epithelium (metaplastic epithelium) similar to ALA, i.e. porphyrin fluorescence was significantly higher in normal tissue compared to Barrett's tissue for both patient groups. The intensity of fluorescence is lower with HAL in the normal epithelium.

FIG. 2 shows the fluorescence intensity of PpIX in the Barrett and normal mucosa in one of the patients receiving HAL. A stronger intensity for the normal mucosa is observed.

### CONCLUSION

The difference in intensity of fluorescence of PpIX induced by HAL in the normal and the Barrett (metaplastic) epithelium permits demarcation of these tissues using PDD.

The lower intensity of fluorescence induced by HAL compared to ALA in the normal and Barrett epithelium is significant. This results in a much lower background fluorescence (i.e. noise) when using HAL in methods of diagnosing pre-cancerous and cancerous conditions in the esophagus. Enhanced demarcation of dysplastic, neoplastic and cancer tissues when using HAL compared to ALA will increase the likelihood of detecting later-stage (i.e. pre-cancerous and cancerous) abnormalities in the esophageal surface.

The results in FIG. 1 show that the inter-patient variation is much less when using HAL. For ALA the standard deviations for the fluorescence intensity of the two tissue types are close to overlapping, whereas for HAL the standard deviations are much smaller. Thus HAL also provides a more reliable diagnostic procedure than ALA when used to distinguish between normal and Barrett's epithelium (metaplasia).

1. A method of photodynamic diagnosis of an abnormality of the epithelial lining of the esophagus, said method comprising the following steps:
(a) administering to a subject a 5-ALA ester, or pharmaceutically acceptable salt thereof;
(b) if necessary, waiting for a time period necessary for the 5-ALA ester or pharmaceutically acceptable salt thereof to be converted into a photosensitizer and achieve an effective tissue concentration at the desired target site in the esophagus;
(c) exposing the epithelial lining of the esophagus to light whereby to photoactive the photosensitizer; and
(d) detecting a fluorescence intensity indicative of said abnormality.

2. The method of photodynamic diagnosis as claimed in claim 1, wherein differences in fluorescence intensity from different tissue types at the target site are detected, said differences in fluorescence intensity being indicative of the presence or absence of said abnormality.

3. The method of photodynamic diagnosis as claimed in claim 1, wherein said abnormality is any one of the following:
metaplasia (Barrett's esophagus);
dysplasia;
neoplasia; and
esophageal cancer (e.g. esophageal adenocarcinoma).

4. The method of photodynamic diagnosis as claimed in claim 1, wherein said abnormality is distinguished from normal tissue in the epithelial lining of the esophagus by its different fluorescence intensity.

5. The method of photodynamic diagnosis as claimed in claim 1, wherein said abnormality is metaplasia (Barrett's esophagus) and is distinguished from normal tissue in the epithelial lining of the esophagus by a lower fluorescence intensity.

6. The method of photodynamic diagnosis as claimed in claim 1, wherein said abnormality is dysplasia, neoplasia or esophageal cancer (e.g. esophageal adenocarcinoma) and is distinguished from normal tissue in the epithelial lining of the esophagus by a higher fluorescence intensity.

7. The method of photodynamic diagnosis as claimed in claim 1, wherein said abnormality is detected in the presence of at least one other abnormal tissue type present in the epithelial lining of the esophagus.

8. The method of photodynamic diagnosis as claimed in claim 7, wherein said abnormality is dysplasia or neoplasia and is distinguished from metaplastic tissue by a higher fluorescence intensity.

9. The method of photodynamic diagnosis as claimed in claim 7, wherein said abnormality is esophageal cancer (e.g. esophageal adenocarcinoma) and is distinguished from metaplastic tissue by a higher fluorescence intensity.

10. The method of photodynamic diagnosis as claimed in claim 7, wherein said abnormality is dysplasia, neoplasia or esophageal cancer (e.g. esophageal adenocarcinoma) and is distinguished from both normal and metaplastic tissue by a higher fluorescence intensity.

11. The method of photodynamic diagnosis as claimed in claim 1, wherein said 5-ALA ester is a compound of formula I:
R²₂N—CH₂COCH₂—CH₂CO—OR¹ (I)
wherein
R¹ represents a substituted or unsubstituted alkyl group; and
R² each independently represents a hydrogen atom or a group R¹.

12. The method of photodynamic diagnosis as claimed in claim 11, wherein said 5-ALA ester is provided in the form of a pharmaceutically acceptable salt, preferably the HCl salt, the nitric acid salt, or a sulfonic acid salt or a sulfonic acid derivative salt, e.g. mesylate, tosylate or napsylate.

13. The method of photodynamic diagnosis as claimed in claim 11, wherein the 5-ALA ester is 5-ALA hexyl ester.

14. The method of photodynamic diagnosis as claimed in claim 1, wherein said 5-ALA ester or pharmaceutically acceptable salt thereof is administered:
(i) topically, for example in the form of a gel (e.g. a gel which comprises a hydrogel or in-situ gelling agent), a spray, a powder, a film, an aerosol or a solution;
(ii) orally whereby to achieve local application to the intended site in the epithelial lining of the esophagus, for example in the form of a high viscosity solution or suspension (e.g. a syrup), a lozenge or a pastille; or
(iii) in the form of a composition which comprises a bioadhesive, preferably a mucoadhesive agent.

15. The method of photodynamic diagnosis as claimed in claim 1, wherein said 5-ALA ester or pharmaceutically acceptable salt thereof is administered in combination with at least one other active component which serves to enhance its photodynamic effect, preferably in combination with a surface penetration assisting agent and/or a chelating agent.
